(19) United States
(12) Patent Application Publication
(10) Pub. No.: US 2002/0018993 A1
(43) Pub. Date: Feb. 14, 2002

(54) Z-CHROMOSOMAL MARKERS DERIVED FROM CHICKEN (GALLUS DOMESTICUS) AND USE THEREOF IN CHROMOSOMAL MAPPING

(76) Inventors: F. ABEL PONCE DE LEON, NORTH OAKS, MN (US); STACY CIUFO, AMHERST, MA (US); JAMES ROBL, BELCHERTOWN, MA (US); SAKTHIKUMAR AMBADY, KERALA STATE (IN); J. ROBERT SMYTH JR., SOUTH AMHERST, MA (US)

Correspondence Address: ROBIN L. TESKIN, SHAW PITTMAN, 2300 M/ STREET, N.W., WASHINGTON, DC, 20037-1128, US

(21) Appl. No.: 09/341,105

(22) PCT Filed: Jan. 2, 1998
(86) PCT No.: PCT/US98/08896

(51) Int. Cl. C12Q 1/68; C07H 21/02; C07H 21/04

(52) U.S. Cl. 435/6; 536/23.1; 536/24.3

(57) ABSTRACT

We have developed a chicken (Gallus domesticus) Z-chromosome-specific DNA library in a phage vector, by means of chromosome microisolation and microcloning. The chromosomal origin, specificity and purity was evaluated by fluorescent in situ hybridization (FISH) on chicken metaphases. Heterologous chromosome painting, using this Z-chromosome-specific probe on turkey (Meleagris gallopavo) metaphases identified its homologous Z-chromosome, under the same stringent conditions as that used in the chicken, indicating a high degree of Z-chromosome sequence homology among these two species. This chicken Z-chromosome library will facilitate the development of Z-chromosome-specific DNA markers that will be useful for genetic mapping in the domestic chicken and related avian species. The Z-chromosome-specific DNA probe will also be useful for studies pertaining to the sex chromosome evolution in avian species.

### CROSS REFERENCE TO RELATED APPLICATIONS

This application claims benefit of priority to PCT/US98/08896, filed Jan. 2, 1998, in turn, to U.S. Provisional Application Serial No. 60/034,410.

### FIELD OF THE INVENTION

The invention relates to novel chromosomal markers derived from chicken and use thereof.

### BACKGROUND OF THE INVENTION

Livestock genome maps have progressed very rapidly in the past few years due to the availability of highly polymorphic DNA markers. But in many species, the maps are not dense enough to facilitate a thorough search for quantitative trait loci (QTLs). This is especially true in the case of the chicken. The chicken haploid karyotype consists of 39 chromosomes that are classified into two categories—the macrochromosomes and the microchromosomes. The largest five pairs of macrochromosomes and the Z-chromosome represent about 55 percent of the total DNA content of the chicken genome. The Z-chromosome covers about 210 cM of the estimated 2500-3,000 cM of the chicken genome map (Levin et al. Genomics, 16:224-230 (1993)).

Knowledge of the genetic composition of the chicken Z-chromosome is limited, in spite of the fact that this chromosome has the most detailed linkage map for this species, largely generated by classical linkage test analyses (Bitgood and Somes, Poultry Breeding and Genetics, 2nd Ed., Crawford R D, ed., Amsterdam: Elsevier, pp. 469-495 (1990)). To date, 19 known loci and 14 genetic markers consisting of 3 chicken middle repetitive sequence element (CRI) markers, 8 random amplified polymorphic DNA (RAPD) markers and 3 microsatellites have been assigned to the chicken Z-chromosome (Bitgood and Somes, (Id.) (1990); Saitoh et al, Chrom. Res., 1: 239-251 (1993); Cheng et al, Poultry Sci., 74: 1855-1874 (1995)).

The avian sex chromosome constitution differs from that of mammals because females are heterogametic (ZW) and males homogametic (ZZ). It has been observed from comparative linkage analyses that some of the sex linked genes in mammals are autosomal in chicken, while some of the sex linked genes in chicken are autosomal in mammals (Bitgood and Somes, (Id.) (1990)). Accordingly, obtaining farther information concerning the Z-chromosome of chickens would be beneficial in identifying sex-linked genes in chickens and related species.

### BRIEF DESCRIPTION AND OBJECTS OF THE INVENTION

Thus, it is an object of the invention to identify novel chromosomal markers from the Z-chromosome of chicken. It is further an object of the invention to use such markers to construct a Z-chromosome specific DNA map and to use such chromosomal markers to identify Z-chromosome homologs in related avian species, e.g., turkey.

In order to develop a dense genetic map for chicken, it is important to generate a large number of polymorphic markers per chromosome (Cheng et al, Poultry Sci., 741:1855-1874 (1995)). One way of achieving this goal is to develop chromosome-specific libraries. Chromosome flow-sorting has been the method of choice for the generation of chromosome-specific libraries in humans (Fuscoe et al, Cytogenet Cell Genet, 43:79-86 (1986)) and in swine (Langford et al, Anim. Genet, 24: 261-267 (1993)). Development of flow-sorted chromosomes is technically demanding and frequently yield preparations which have some degree of contamination with other chromosomes (Hozier and Davis, Anal. Biochem, 200: 205-127 (1992)).

A more effective and direct way of generating chromosome-specific DNA libraries is by chromosome microisolation and microcloning of the chromosome of interest. Chromosome specific libraries generated by chromosome microisolation have been used in swine (Ambady et al, (unpublished data)), cattle (Ponce de León et al, Proc. Natl. Acad. Sci., USA, (in press) 1996)), and chicken (Li et al, Proc. of the 10th Eur. Colloq. on Cytogenetics of Domestic Animals, Utrecht Univ., The Neth., p. 11, August 18-21 (1992)) genetic mapping studies in order to develop maps for particular chromosomes. Generation of polymorphic markers from chromosome-specific libraries for all of the 8 pairs of the chicken macrochromosomes will enable saturation of about 55-70% of the chicken genome. Chromosome-specific DNA can also be used as heterologous chromosome painting probes in closely and distantly related species for comparative genome analysis, study of chromosomal evolution, and for identifying gross chromosomal abnormalities.

This application, in particular, provides a chicken Z-chromosome-specific DNA library, Z-chromosomal markers and use thereof as probes to identify the Z-chromosome homolog in related species, such as turkey.

### BRIEF DESCRIPTION OF THE FIGURES

FIG. 1 shows amplification of microsatellite markers by PCR and identification of polymorphisims.

FIG. 2 shows a genetic map constructed using the identified microsatellite markers.

FIG. 3 shows dinucleotide repeats present in the identified microsatellite markers.

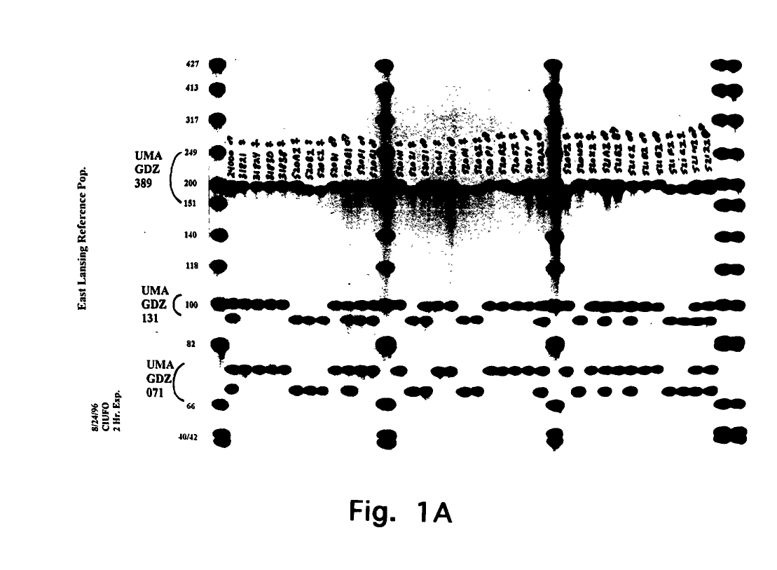

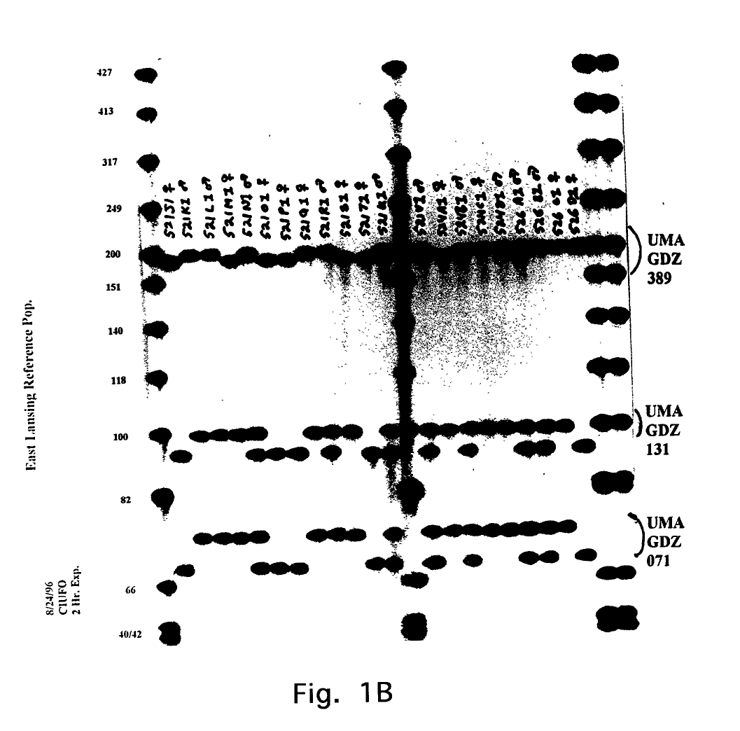

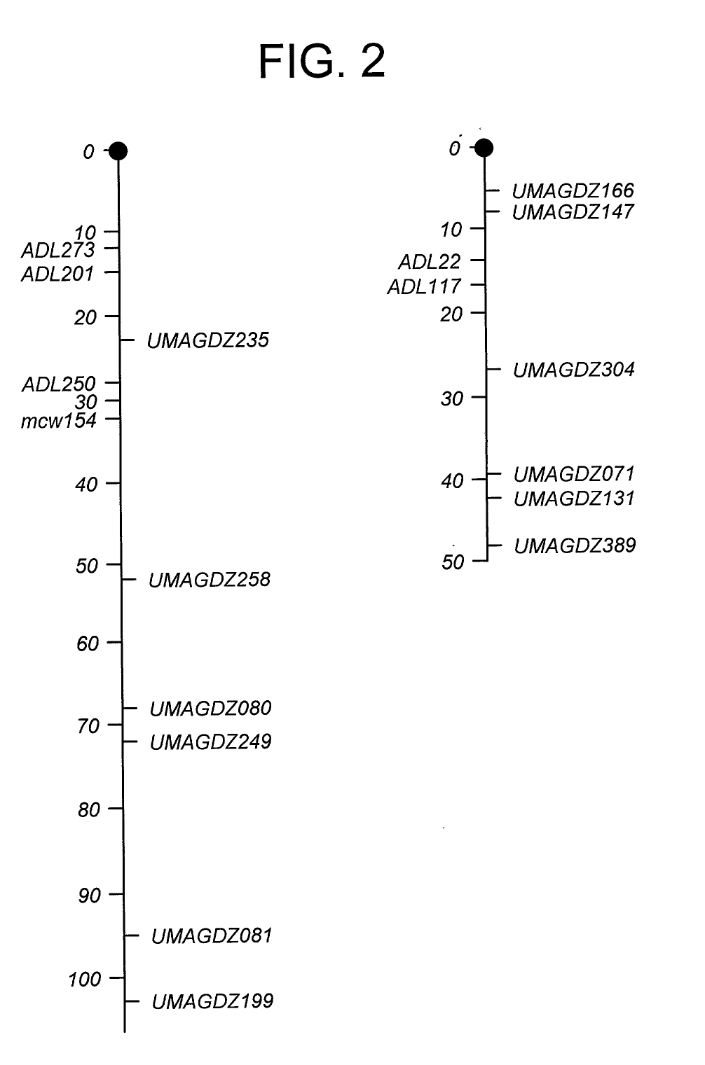

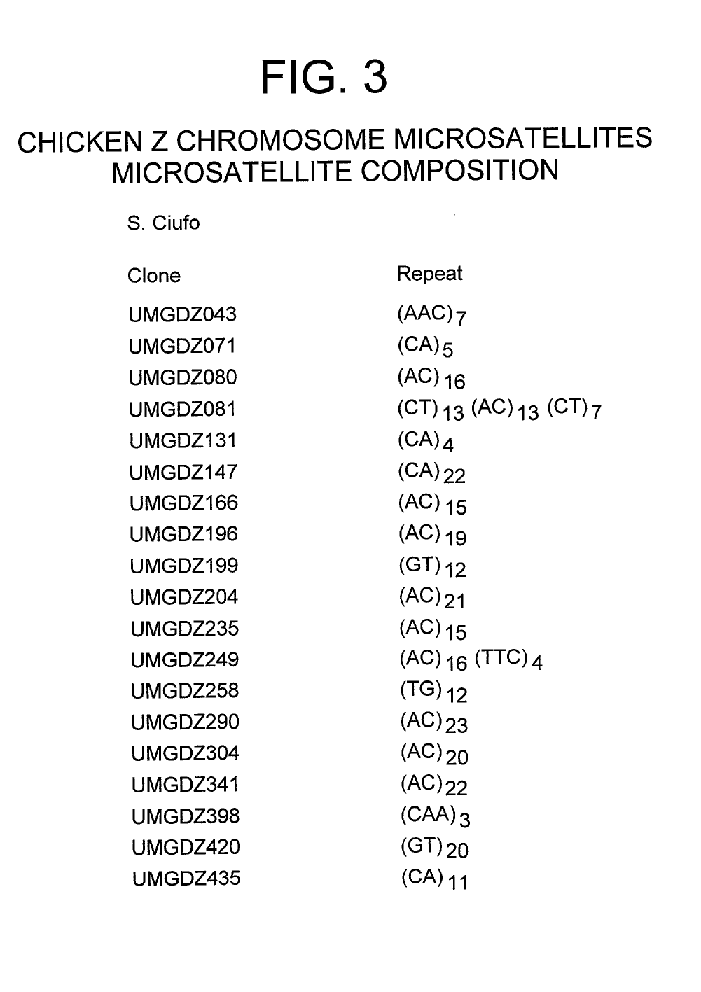

### DETAILED DESCRIPTION OF THE INVENTION

Microisolation and Microcloning

Chicken metaphases were prepared from chicken fibroblast cultures following standard procedures, fixed briefly for 5 minutes each in 9:1, 5:1 and 3:1 methanol: acetic acid and dropped on clean coverslips. Chromosome microisolation and cloning was performed following the procedure described by Ponce de León et al (Proc. Natl. Acad. Sci. USA (in press) (1996)). Briefly, twelve copies of the chicken Z-chromosome were microisolated and transferred to clean siliconized coverslips. Proteinase-K digestion, phenol-chloroform extraction, Sau3AI (50U/μl, New England Biolabs) digestion and ligation to custom prepared Sau3AI adaptors were performed in a nanoliter drop. Ligation products were digested with BgII enzyme (Promega, 10 units/μl) to cleave off the adaptor dimers that form during the ligation process.

The ligation product was PCR amplified and 10 μl of the amplified product was run on an agarose gel to determine the size of the amplified products. A 2 μl volume of this original amplification was labeled by PCR, using biotin-16-dUTP (Boehringer Mannheim). The purity, specificity and origin of the DNA fragments was determined by FISH on chicken metaphases following the procedure described by Ponce de León et al (Proc. Natl. Acad. Sci. USA (in press) (1996)). The remainder of the PCR product was digested with Sau3AI and passed through a Microcon 30 (Amicon Inc.) spin column to cleave and remove the flanking adaptors respectively.

In order to produce a chicken Z-chromosome-specific phage library, the digested DNA was cloned in a lambda ZAP Express vector (Stratagene) and packaged using Gigapack II Gold packaging extract (Stratagene). The library was amplified by plate lysate method following the manufacturer's protocol and stored at −70° C. in 7% DMSO and 0.3% chloroform. Average size of library inserts was determined by PCR amplification of 30 randomly picked clones using the T3 and T7 priming sites flanking the insert.

### Fluorescent in situ Hybridizations

The Z-chromosome-specific DNA fragments were fluorescently labeled by PCR with biotin-16-dUTP (3:1 ratio of dTTP:biotin-16-dUTP) and passed through a Sephadex G-50 column to remove unincorporated nucleotides. The protocol described by Ponce de León (Proc. Natl. Acad. Sci., USA (in press) (1996)) was followed. Briefly, 200 nanograms of labeled Z-chromosome specific DNA was mixed with 6 μg of chicken competitor DNA (average size 200-400 bp) and 5.8 μg of salmon sperm DNA (average size 200-400 bp), precipitated and resuspended in 12 μl of hybridization buffer consisting of 50% deionized formamide, 1×SSC and 100% dextran sulphate to achieve a final DNA concentration of 1 μg/μl. The hybridization mix was denatured at 75° C. for 5 minutes and reannealed at 37° C. for 10 minutes and deposited on denatured (70% formamide, 2×SSC at 70° C. for 2 minutes) chicken or turkey metaphases, mounted, sealed with rubber cement and incubated in a humidified chamber at 37° C. for 18 to 20 hours. The slides were washed in 50% formamide/2×SSC at 42° C. for 15 minutes and 0.1×SSC at 60° C. for 15 minutes. Blocking was done using 2% blocking reagent (Boehringer Mannheim) and the signals were detected using avidin-FITC (5 μg/ml, Vector labs) in 1% blocking solution. Slides were washed in 4×SSC/0.1% Tween-20 for 15 minutes at 42° C., stained for 10 minutes in propidium iodide (400 ng/ml in 2×SSC) and rinsed for 5 minutes in 2×SSC/0.01% Tween-20. Slides were mounted in p-phenylenediamine-11 (PPD-11) antifade and observed under a Zeiss Axioskop fluorescent microscope.

### Results

A chicken Z-chromosome specific DNA cocktail was developed by chromosome microisolation, Sau3AI digestion, adaptor ligation and PCR amplification. The amplified DNA fragments ranged in size from 400 bp to 1600 bp with the bulk of the DNA in the 500-1000 bp range. The origin, specificity and purity of the chromosomal DNA fragments was verified by FISH after PCR labeling of a small fraction of the DNA cocktail. The probes showed specific hybridization signal on a medium sized submetacentric chromosome identified as the Z-chromosome based on its morphology and G-banding pattern. After having confirmed the origin and purity of the preparation, the adaptors flanking the inserts were removed by Sau3AI digestion and column purification. Cloning was performed using equimolar ratios of the inserts to the vector ends (lambda ZAP Express, Stratagene). The original library consisted of a total of 8.48×10⁵ plaques representing about 14 chicken Z-chromosome equivalents. The final titer of the amplified library was 1.2×10¹² pfU/ml.

Thirty random plaques were selected and the inserts PCR-amplified using the T3/T7 priming sites flanking the inserts. The average insert size was about 1,000 bp (data not shown). This library was screened to identify microsatellite containing clones to increase the marker density of the chicken Z-chromosome genetic linkage map.

### Heterologous Painting of Turkey Metaphase Chromosomes

The labeled chicken Z-chromosome-specific DNA fragments were used to perform FISH analysis on turkey metaphase chromosomes following the procedure described previously. Washes at the same stringency showed strong hybridization signals on a medium-sized submetacentric chromosome in turkey metaphases (data not shown). This chromosome was identified as the Z-chromosome homolog in the turkey. The obtained results indicate that the chicken and turkey Z-chromosome sequences are highly conserved. The red-legged partridge Z-chromosome has also been shown to be homologous to the chicken Z-chromosome (Dias el al, Proc. of the XXIV Int. Cont. on Anim. Genet., Prague, Czech. p. 133 (Jul. 23-24, 1994)). These results are similar to the FISH results obtained when the bovine X-chromosome painting probes were used on sheep and goat chromosomes (Ponce de Le\acute{6}n el al, Proc. Natl. Acad. Sci., USA (in press) (1996)) and with human X-chromosome probes on a wide range of mammalian species (Schertan el al, Nat. Genet., 6:342-347 (1994)) indicating the high degree of sex chromosome conservation among all the mammalian species studied. Solinas-Toldo et al (Genomics, 27: 489-496 (1995)) have previously shown that human chromosome-specific painting probes could identify chromosomal segments in bovine that are homologous to specific human chromosomes. It is expected based on our results that chicken chromosome painting probes can similarly be used in closely and distantly related avian species to identify gross chromosomal rearrangements such as translocations and duplications that have occurred during avian evolution. Since the chicken Z-chromosome sequences are highly conserved in the turkey, the chicken Z-chromosome-specific microsatellite markers should be particularly useful for genetic mapping in turkey.

### Conclusions

Genetic and physical mapping of human and animal genomes has been greatly facilitated by the use of chromosome specific DNA libraries. Mapping with libraries specific to a chromosome or chromosomal region increases marker saturation by reducing the gaps resulting from a purely random shotgun approach. This study was undertaken to construct a genetic and physical map of microsatellites on the chicken Z chromosome. This chromosome is the fifth largest in the chicken genome, comprising about 8% of the total. Notwithstanding its size, very few microsatellites have been assigned to it. DNA originating from the chicken Z chromosome was previously isolated and reported. This was used to construct a small insert library in Lambda ZAP Express, representing 14 chromosome equivalents. This library was screened for microsatellites with an (AC) 12 oligo, and positive clones were isolated. Confirmation of the presence of the microsatellite, as well as its approximate location along the cloned fragment was accomplished by PCR amplification. Clones with adequate flanking regions were sequenced, and primers for 19 microsatellites were constructed. These primers were used to genotype individuals from the East Lansing Poultry Reference Population and a linkage map was constructed. Fourteen markers were scorable and polymorphic in this population. The resulting map contains 12 markers in two linkage groups spanning 90 Cm and two unlinked markers. The physical location of each marker was established by fluorescent in situ hybridization (FISH). Preliminary results with four markers allowed the assignment of one linkage group to the long arm of the Z chromosome, and one to the short arm.

The following nucleic acid sequences are microsatellite markers identified by the above methods. As discussed supra, these markers are useful for genetic mapping and for study of the sex chromosome structure in avian species. Also, such markers should enable the identification of genes encoding desirable traits, e.g., genes involved in growth rates, and for identifying sex-linked genotypes.

### EXAMPLE

The specific Gallus domesticus microsatellite markers identified are set forth below. As noted, these DNA markers will be useful for genetic mapping of domestic chicken as well as related avian species and for studies pertaining to evolution of the sex chromosome in avian species.

| | | |
|---|---|---|
| 1 | gatcactttc cctaatattc ttgtgtttct tgtttgttga cctgtaatgc | SEQUENCE 1 (43. Seq) |
| 1 | agttctgagt tttggaaagg aactaattaa gaccagagga gagataattt | |
| 101 | tcttttatca aaaaacaaac aaacaaacaa aaaaacgaat tcttaccact | |
| 151 | ttacaaaaat tttccatttt gaaggccagt acagccatag cattcatcta | |
| 201 | ctttttgctt tggat | |
| 1 | gatcaggtgg cctgtagtag acaacaacaa caatggggtg ccctttgttg | SEQUENCE 2 (71. Seq) |
| 51 | ccttagtctc taactcgcac ccacacacac tttcaagttg cttgtggcca | |
| 101 | ttcttcaggg acagttcttc acaatctatt cctttcctga tgtagaaggc | |
| 151 | gtcacctcct cccctcctgc ctcgtttgtc ccttctaaac tgcaggtatt | |
| 201 | agtattgata gctaaggtca agtcatggga accatctcac caggtttcag | |
| 251 | tgttggcaac tatgttatgc tttcttagga gcatggtggt tccaactctt | |
| 301 | ccctgcttat ttcccaagct gtgtgtgatg gtaggatagc attcaagtgg | |
| 351 | gaggagccta tcggcttttt ggaggtactc ctaaatccct gatattcccc | |
| 401 | tgattcccgt acttcttcct tgccaagggc ccgccaatgc atagttcaat | |
| 451 | ttctcatgca gacgctaagg aaaggtggac cc | |
| 1 | gatcgtatgt atttttttac ataggataga aaatggccaa taggaaataa | SEQUENCE 3 (80 Seq.) |
| 51 | gacagtacag ctactaagaa agaaacacaa ttacacacac acacacacac | |
| 101 | acacacacac acacatttga aaaacgcgct gcacagcagt gtgggtattt | |
| 151 | tttcacaaga gagacacact ctacagtaca cagccagctc tactttgtcg | |
| 201 | cacagtctca gtgtgtgttt gccaacagga cgcggttcac agggagatat | |
| 251 | tgtcctcttg tgtgtgtgga gacacagaga cagag | |
| 1 | gatcccctgg aggaagggca atggcaaccc actccagtat tcttgcctga | SEQUENCE 4 (81. Seq) |
| 51 | agaataccat ggtcagtttt gcctcctggg ctatagtcca tggggttgca | |
| 101 | aagagtcagg catgactgag cgactctctc tctctctctc tctctctctc | |
| 151 | acacacacac acacacacac acacacggcg tctctctctc tctctataca | |
| 201 | tataggctgt gtgtctcgct attctcacat gagggaaact catatctagc | |
| 251 | acgtggcaca aatattgttt gtggctctca caaaagacat gtgggcgcac | |
| 301 | aaaggtcccc ccccggtgga tacancgcct tggtttttta taacccaagc | |
| 351 | ctgtg | |
| 1 | gatcacatat gtaaactagg gaattgcata ataagattaa atgtaggtgt | SEQUENCE 5 (131 Seq) |
| 51 | agaacgtggc atgaaggaag gtagaattag gtggtaccta tctcttctga | |
| 101 | aacaaactga gaatcctact accaatcaac atattctaca taccacacac | |
| 151 | acattttttc tcgagtaaaa tataaactaa tgagaaactt ccctag | |
| 1 | gatcccaagc aacacatagn cagacaatca cacacacaca cacacacaca | SEQUENCE 6 (147. Seq) |
| 51 | cacacacaca cacacacaca cacatcctct ccccacaata catcccgaga | |
| 101 | ggggggagag acactctctc tccctctcta taggggagac ccggagagct | |
| 151 | ggctctgttg tctctctaca ccggacatac agtggagcac atctcacact | |
| 201 | tgtgtctttg tctctctaca ccggacatac agtggagcac atctcacact | |
| 251 | tgtgtctcta tctctccctg tccctgttga tccatctctc ttcacacatc | |
| 301 | tctccagatc ttagcgctag agtctcctgt cttctctctg cgcaatttgt | |
| 351 | gtgatagaga cacctgatat gttgtgtggg ggagacatct gtgtgtctct | |
| 401 | gtgtcatccc agaggatttt tctctcccac acttagaggc cttctcaaga | |
| 451 | gatgggaggt tttaatgggg tgtg | |
| 1 | gatcattctt ctgtttccca ttctaatggg aattctccac acacacacac | SEQUENCE 7 (166. Seq) |
| 51 | acacacacac acacacacat cttcttcccc ttacatggaa aaaaatcctc | |
| 101 | cacacccctg gacactgatt actctccctc ttcccagaga gagatc | |
| 1 | gatcccctag agaagggaat ggctactcac tccagtattc ttgcctggag | SEQUENCE 8 (196. Seq) |
| 51 | aattccgtgg tcagaggagc ctggaaggct ataatccata gagtcgcaag | |
| 101 | agtcagacag gactgagtga ctaacacaca catgcacaca cacacacaca | |
| 151 | cacacacaca cttgctctag ggagaggcat agagatgtaa tctctcctaa | |
| 201 | aatgggggtg gcgatggccc ctgcggccaa gtaatcgcca cacatgcgta | |
| 251 | ttccccttaa gattgggtta ggcctccctt atgaggagag accagggaga | |
| 301 | gaatgggctc tctctctctc tcactcccca accgagtaag tggtaaaaaa | |
| 351 | ggttttcctg gattacaatt ttggtgttac agaattggaa aaaaatattt | |
| 401 | ttggggctcc cccctcagtt ta | |
| 1 | ctagcaaaaa cacccccaca agttatgaaa acaacggctt aatatagtaa | SEQUENCE 9 (199. Seq) |
| 51 | tgtgtgtgtg tgtgtgtgtg tgttgcacac cacagttttc tctgatactc | |
| 101 | aaacctctct ctttctctac aggggccccc cataacacag cggctgagat | |
| 151 | gtgtgacggg aaggcgtggc cttttacaca tttgtggtat ggtctgccaa | |
| 201 | ggccccctat tgccccccac aactacggag atacactagg ggcgacccgc | |
| 251 | aggcgcgcga cccccaggtg gggccccgag | |
| 1 | ctttaggagg ttctctcgag taagcttttt ggatttcttt ggttcccaag | SEQUENCE 10 (204. Seq) |
| 51 | catcacatgg tacaggcagt cacacacaca cacatacaca cacacacaca | |
| 101 | cacacacaca cactcctctc cccacaatac ataccgagag gggggagaga | |
| 151 | cactctctct ccctctctat agggggagcc ccacagagct ggctctgttg | |
| 201 | tctctctcca ccggacatac agtggagcac atctcacact tctgtctcta | |
| 251 | tctctccctg cccctgtgac atccatctct cttcacacaa tctcacccag | |
| 301 | gatcttagcg ctagagaccc cctgtccttc ttctcctggg gaaatttttt | |
| 351 | gtggataaga gacacccgat atattggtgt gggggagaac atcttgtgag | |
| 401 | gtctctgttg tgccatccca acaggaattt ttatctcccc cacaattaga | |
| 451 | ggcccctcct caagagtgtg tgagggtt | |
| 1 | gatcacagat gtatgtattt ttttacatag gatagaaaat ggacaatagg | SEQUENCE 11 (235. Seq) |
| 51 | aaataagaca gtacagctac taagaaagaa cccacattta cacacacaca | |
| 101 | cacacacaca cacacacaca agtgtttaat ccgctgcaca gcattgtgga | |
| 151 | catttttaca caagagagac acactctaca gtttgcgccc agctctag | |
| 1 | gatcattctt ctgtttccca ttctaatgga attctccaca cacacacaca | SEQUENCE 12 (249. Seq.) |
| 51 | cacacacaca cacacactct tctttctcct gacatggaaa aatctccccc | |
| 101 | acaccccggg acactgattt ctctccctct ccccaacact gtgagcaaga | |
| 151 | ggagtttatt ttgtgtgtgt cactcttcca gggagagaga gatc | |
| 1 | ctaggcatcg gttgggaggt ggtgagtaat tacttgtctg acattagtcc | SEQUENCE 13 (258. Seq) |
| 51 | tgtaacattg ggtgtgtgtg tgtgtgtgtg tgtgtattcc ccttgggaat | |
| 101 | tggttttctc aaccacaagt tcttcttttt tttttttctc cccccttttc | |
| 151 | ttctgaaaat aagtacttgg ggggtttccg ccccccccgg taaataaaat | |
| 1 | ctagtggctc ccaagcaaca catagccaga caacacacac acacacacac | SEQUENCE 14 (290. Seq) |
| 51 | acacacacac acacacacac acacacactc ctctccccac aatacatccc | |
| 101 | gagagggggg agagacactc tctctccctc tctatagcgg gagccccaca | |
| 151 | gagctggctc tgctgtctct ctacaccgga catacagtgg agcacatctc | |
| 201 | acattcgtgt ctctatctct ccctgcccct ggtgacatac atctctcttc | |
| 251 | acacatctca ccaggtctga gcgctagagt ctcctgtctt ctctctgcgc | |
| 301 | aatatttgtg atagagacat ctgatatatt gtgtgtggga gacatcttgt | |
| 351 | gagtctctgt gtgcatccca gaggattttt atctccccac actag | |
| 1 | gatccatgaa aactttccga gttgtattgt ctaggtgaaa acacacacaa | SEQUENCE 15 (309. Seq) |
| 51 | acacacacac acacacacac acacaacagg gagatgagtc ttgcaagaga | |
| 101 | ataggggaga gttatgtcac caagtctggt gaggtatata gcgtataggg | |
| 151 | agccaacatg tcagacatct gatgtgctaa gattaacatt ttattttatt | |
| 201 | taatgtgtga gatctcatat agcggctctt cttatatatg acgtctcgca | |
| 251 | atgtctcttt atgtgtgtta ttctctgagc ccctgggaga tatctgtcat | |
| 301 | cagagagaag agacatacac atacaggggt tatatatttt ctccctgtgt | |
| 351 | gtggagatgg agggtatttt ggacaagctc aacactcatt ggctcccaga | |
| 401 | gagagaaaag gagcaactgt tgcacccggg gctctgtagc tgggatc | |
| 1 | caattgggta catctacctg gtaccccacc cgggtggaaa atcgcatggg | SEQUENCE 16 (341. Seq) |
| 51 | cccgcggcgg ttctaggaag tactctcgag aagcttttgg gttctttggg | |
| 101 | tcccaagcag cacatggaca ggcaatcaca cacacacaca cacacacaca | |
| 151 | cacacacaca cacacacaca ctcctctccc cacaatacat cccgagaggg | |
| 201 | gggagagtca ctctctctcc ctctctatag ggggcgcccc taagagctgg | |
| 251 | ctctgttgtc tatctacacc gcacatacaa tggagcacaa ctcacactag | |
| 1 | gatcaaagca tggaggtcat gccaggcact gaacaaaatg gtagagagtg | SEQUENCE 17 (398. Seq) |
| 51 | attctatgac tgactaagac ctcatgcaac aacaagtgaa gagtcacaac | |
| 101 | tgcaaacaga agtacaactt agcaaatcct attttcagga aacactaaac | |
| 151 | cgtaatactt gcacgatttt ttctttaata cagtaataat tcttttagaa | |
| 201 | tttggatata tcttttaaga tacatatttg tctaaatacc aaggcaggat | |
| 251 | atgagcataa aatagctaag gttagctatg gtgttatatt taagaagacc | |
| 301 | acagagcaat aggagcatac ttttcttggg gtagaagggg cccttaaagg | |
| 351 | tcacctag | |
| 1 | ctagccacat cctataactc cactccacct ttaatcctga tttctgtgtc | SEQUENCE 18 (420. Seq) |
| 51 | tcttctctaa cctctatggc ctttctctaa agttccccaa tatcaacaat | |
| 101 | ccttttcccc actgggacct ccagtttatt gattctacca tgtcactatc | |
| 151 | catggtcaac cacttgtggt attataggat gtcgcgtgtg tgtgtgtgtg | |
| 201 | tgtgtgcatg tgtgtgtgct tgggtgtcag agagttccaa tctgggggac | |
| 251 | ctatggtttg taaacaacag gtctcttgcc aaggaagat | |
| 1 | ctagcgctcg tgcccctgca gttcgacact cagtggctcc tccacacaca | SEQUENCE 19 (435. Seq) |
| 51 | cacacacaca cacatcaata tatatataga tagatagata gatagaggag | |
| 101 | caatataagt ggcttctcta tttccagcat gttttgaaga gcataaactc | |
| 151 | aacagagtat atataaatct gatgtgaccc atgtcatctg ctacagcatg | |
| 201 | agagggggta gtgatc | |

What is claimed is:

1. A Z-chromosomal marker DNA selected from the group consisting of Sequence I (43. Seq), Sequence 2 (71. Seq), Sequence 3 (80. Seq), Sequence 4 (81. Seq), Sequence 5 (131. Seq), Sequence 6 (147. Seq), Sequence 7 (166. Seq), Sequence 8 (196. Seq), Sequence 9 (199. Seq), Sequence 10 (204. Seq), Sequence 11 (235. Seq), Sequence 12 (249. Seq), Sequence 13 (258. Seq), Sequence 14 (290. Seq), Sequence 15 (309. Seq), Sequence 16 (341. Seq), Sequence 17 (398. Seq), Sequence 18 (420. Seq), and Sequence 19 (435. Seq).

2. A Z-chromosomal DNA library that contains at least one DNA sequence according to claim 1.

3. A method of using at least one Z-chromosomal DNA according to claim 1 for genetic mapping.

4. The method of claim 3, wherein the genetic mapping is effected to construct a Z-chromosome specific DNA map.

5. The method of claim 3, wherein the Z-chromosome DNA map is that of an avian species selected from the group consisting of chicken, turkey, partridge, duck, guinea hen, and goose.

6. The method of claim 4, which is used to identify gross chromosomal rearrangements.

7. The method of claim 6, wherein said chromosomal rearrangement comprises a translocation, deletion or duplication.
